(19) United States
(12) Patent Application Publication
(10) Pub. No.: US 2005/0261641 A1
(43) Pub. Date: Nov. 24, 2005

(54) Method for ophthalmic administration of medicament

(76) Inventors: Mark P. Warchol, Kalamazoo, MI (US); Praveen Tyle, Princeton, NJ (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/659,813

(22) Filed: Sep. 11, 2003

Related U.S. Application Data
(60) Provisional application No. 60/413,959, filed on Sep. 26, 2002.

(51) Int. Cl. A61M 35/00

(52) U.S. Cl. 604/294

(57) ABSTRACT

A method for treatment or prevention of a disease or disorder of an eye comprises (a) charging a dispenser with a suitable liquid medicament, (b) disposing the dispenser in operative juxtaposition with the eye, and (c) actuating the dispenser to release a therapeutically effective amount of the medicament into the eye. The dispenser comprises an electrically energizable droplet generating device, such as a thermal resistor bubble jet device, together with means for electrically energizing and means for actuating the device. The device, when actuated, is adapted to issue droplets of the liquid medicament at a rate of about 1 to about 300 μl s⁻¹ whereby a therapeutically effective amount of not more than about 50 μl of the medicament is released into the eye in not more than about 1 second. The dispenser further comprises a standoff configured to engage a facial surface proximal to the eye, thereby placing the dispenser in operative juxtaposition with the eye.

This application claims priority of U.S. provisional application Ser. No. 60/413,959 filed on Sep. 26, 2002.

### FIELD OF THE INVENTION

The present invention relates to delivery to eyes of metered doses of medicaments, more particularly liquid formulations of drugs useful in treatment or prevention of ophthalmic diseases or disorders.

### BACKGROUND OF THE INVENTION

Problems exist in the art of administering pharmaceutical compositions to eyes for treatment or prevention of ophthalmic diseases and disorders, and for other purposes. Various devices have been proposed or developed for ophthalmic administration of drugs, including those disclosed in the patents identified individually below and incorporated herein by reference.

U.S. Pat. No. 4,834,728 to McKenna.

U.S. Pat. No. 5,201,726 to Kirkham.

U.S. Pat. No. 5,578,021 to Cornish.

U.S. Pat. No. 5,588,564 to Hutson & Demangus.

U.S. Pat. No. 5,894,841 to Voges.

U.S. Pat. No. 6,033,389 to Cornish.

However, present methods each appear to suffer one or more of the following drawbacks:
(a) inadequate controllability of direction, distance and timing of projection of substances into an eye;
(b) poor suitability for self-administration;
(c) imprecise metering of dosage;
(d) requirement for complex drug formulations; and
(e) poor adaptability to low volumes of drug formulation.

Printing technologies offer droplet generation devices that have been proposed for delivery of drugs and personal care products to a subject. For example, U.S. Pat. No. 5,518,179 to Humberstone et al., incorporated herein by reference, proposes, in FIG. 19 thereof, generating droplets of a fluid by electromechanical or electroacoustic means, for example by electrically actuated vibration of a membrane to which the fluid is supplied, and directing the droplets on to skin of a subject, as a means of application of a personal care product.

Above-cited U.S. Pat. No. 5,894,841 discloses a dispenser comprising a reservoir that holds a physiologically active substance, and a droplet ejection device that controllably issues droplets of the substance from ejection orifices upon actuation. The disclosure of U.S. Pat. No. 5,894,841 is directed especially to an inhaler, adapted for pulmonary delivery of drugs, but also encompasses, as illustrated in FIG. 5 of the cited patent, a hand-held apparatus adapted for topical delivery. Actuation to generate droplets can be by means of a piezoelectric crystal or thermal resistor bubble jet device such as used in an ink jet print head.

U.S. Pat. No. 5,368,582 to Bertera, incorporated herein by reference, discloses a device having pump elements for introducing fluid material into a subject's eye. Among pump elements disclosed are thermal-expansion jet pumps of the kind used in ink jet printers. The pump elements are disposed on a spectacle frame worn by the subject, or in one embodiment as illustrated in FIG. 9 of the cited patent, on stalks projecting from a ring-like support having legs that permit the support to rest away from and centered over the eye. This embodiment is said to be useful for administering fluid to the eye during surgery requiring eye irrigation.

The device of above-cited U.S. Pat. No. 5,368,582 is said to be capable of delivering individual droplets of a size of the order of 200 picoliters (0.0002 μl) at a rate of 1 to 2000 Hz, illustratively 100 Hz. This can be calculated to provide a rate of fluid delivery to the eye of about 0.0002 to about 0.4 μl s⁻¹, illustratively about 0.02 μl s⁻¹, i.e., approximately 1 μl per minute. Such a delivery rate is stated to be an adequate rate for irrigation purposes. However, it is inadequate for delivery of a medicament in a form of a liquid composition comprising a drug, where it is desired to place a volume of about 1 to about 50 μl, more typically about 5 to about 30 μl, of the composition in an eye within about one second or less.

The need for delivery within such a short space of time arises in part from a tendency of the subject to blink the eyes. If delivery is more prolonged, the probability increases of one or more blinks occurring during delivery, thereby wasting medicament and resulting in underdosing of the eye.

There remains a need, therefore, for a highly controllable, precisely meterable delivery method for delivery of volumes of a liquid medicament in excess of about 1 μl within about 1 second or less.

This need is especially great where the medicament to be delivered is an agent for reduction of intraocular pressure, a major factor in glaucoma and other serious disorders of the eye.

### SUMMARY OF THE INVENTION

There is now provided a method for treatment or prevention of a disease or disorder of an eye, the method comprising (a) charging a dispenser with a suitable liquid medicament, (b) disposing the dispenser in operative juxtaposition with the eye, and (c) actuating the dispenser to release a therapeutically effective amount of the medicament into the eye. The dispenser used in the method of the invention comprises an electrically energizable droplet generating device together with means for electrically energizing and means for actuating the device. The droplet generating device, when actuated, is adapted to issue droplets of the liquid medicament at a rate of about 1 to about 300 μl s⁻¹ whereby a therapeutically effective amount of not more than about 50 μl of the medicament is released into the eye in not more than about 1 second. The dispenser further comprises a standoff configured to engage a facial surface proximal to the eye, thereby placing the dispenser in operative juxtaposition with the eye.

Suitable electrically energizable droplet generating devices are well known in the printing art, and include, for example, thermal resistor bubble jet devices and piezoelectrically actuated ink jet devices. A bubble jet device is presently preferred.

In a preferred embodiment the dispenser useful in the method further comprises means for selecting prior to actuation thereof at least one of (a) a rate and (b) a duration of issuance of the medicament. Accordingly there is now provided an apparatus for dispensing a liquid medicament to an eye, the apparatus comprising:
an electrically energizable droplet generating device adapted to issue droplets of the liquid medicament at a rate of about 1 to about 300 μl s⁻¹;
means for electrically energizing the device;
means for actuating the device;
a standoff configured to engage a facial surface proximal to the eye whereby the dispenser can be disposed in operative juxtaposition with the eye; and
means for selecting prior to actuation at least one of (a) a rate and (b) a duration of issuance of the medicament.

### BRIEF DESCRIPTION OF THE DRAWING

FIG. 1 is a schematic drawing in section view of a dispenser useful in the method of the invention.

### DETAILED DESCRIPTION OF THE INVENTION

The method of the invention is described herein with particular reference to an apparatus comprising a thermal resistor bubble jet device; however, it will be recognized that any electrically energizable droplet generating device that is capable of delivering droplets at a rate of about 1 to about 300 μl s⁻¹ can be substituted.

The method of the invention comprises (a) a step of charging a dispenser as described herein with a liquid medicament suitable for treatment or prevention of a disease or disorder of an eye, (b) a step of disposing the dispenser in operative juxtaposition with the eye, and (c) a step of actuating the dispenser to release a therapeutically effective amount of the medicament into the eye.

In the first step, that of charging the dispenser, the medicament is placed in fluid connection with the bubble jet device. If desired, the medicament can be fed to the dispenser from an external container or reservoir. Alternatively, a refillable vessel is permanently housed in the dispenser, in which case the dispenser is charged by adding a volume of medicament to the vessel. Preferably, however, the dispenser is charged by means of a replaceable cartridge reservoir. In this case the dispenser comprises a housing having a cavity adapted to accept a replaceable cartridge. The cavity can be externally exposed on the housing or can be wholly internal, with access means to enable cartridge replacement. When correctly installed in the cavity, the cartridge is automatically disposed in fluid connection with a conduit that feeds the medicament from the cartridge to the bubble jet device.

The second step involves disposing the dispenser in operative juxtaposition with the eye. By “operative juxtaposition” herein is meant placement and orientation of the dispenser relative to the eye such that droplets issuing from the bubble jet device are directed into the eye from a suitable distance. To ensure operative juxtaposition, the dispenser is provided with a standoff configured to engage a facial surface proximal to the eye. One or more parts of the facial surface, including cheek, eyebrow and nose, can be engaged by the standoff. In a preferred embodiment the standoff takes the form of a cup having a rim contoured to engage the facial surface, more or less completely surrounding and enclosing the eye to be treated. Such a cup can be fabricated from a material providing sufficient flexibility and resilience to conform to the shape of the circumocular portion of a subject's face without causing discomfort. However, the cup should not be so flexible as to permit excessive latitude in disposition of the dispenser, i.e., disposition other than in operative juxtaposition as defined above.

In the third step, the dispenser is actuated. By actuation is meant putting the bubble jet device in an operative condition such that droplets of the medicament are or can be expelled from the device. Typically actuation involves operating a switch to connect the bubble jet device to electrical energizing means. In some embodiments override controls can be present in the dispenser, for example connected to a sensor that detects whether the dispenser is correctly positioned and oriented with respect to an eye, and such controls can prevent release of the medicament even when the dispenser is actuated.

A dispenser used in the method of the invention comprises (a) a thermal resistor bubble jet device; (b) means for electrically energizing the bubble jet device; (c) means for actuating the bubble jet device; and (d) a standoff as described above.

The bubble jet device can be of any design known in the printing art. A typical thermal resistor bubble jet device comprises one to a plurality of coaxially divided nozzles each having an orifice on an outer side and a chamber on the opposing inner side. A thin film resistor is located in the chamber adjacent to each nozzle. A liquid is supplied to the chamber via a conduit from a reservoir. When a resistor is electrically energized, it heats rapidly. Within a few microseconds, liquid in the chamber in contact with the energized resistor vaporizes and forms a bubble, which grows rapidly and imparts pressure and momentum to the liquid. Some of this liquid is ejected as a droplet from the nozzle orifice at a velocity typically exceeding 10 m s⁻¹. The ejected volume of liquid is immediately and automatically replaced by flow into the chamber from the conduit. Up to 6000 or more droplets per second can be generated by such a device when used in printing.

The design of the bubble jet device in a dispenser useful in the present invention is preferably modified from that used in the printing art. In a bubble jet printer, the nozzles are typically arranged in a rectangular matrix of, for example, 12 or 24 nozzles, but the bubble jet device of a dispenser useful herein can have any number and arrangement of nozzles, preferably a single nozzle or a small plurality of nozzles arranged in close proximity to each other, for example in a circle. Larger droplets than those used in printing may be desired; this can be achieved by increasing nozzle orifice diameter. Nozzle orifices can differ in diameter one from another so that droplet size can be controlled by selecting which nozzles are used for droplet generation. The size of droplet generated is predetermined for a given liquid, nozzle, and control setting, but the frequency of droplet production can be controlled with great precision.

The electrical energizing means is preferably a battery contained entirely within the dispenser, for example inside a housing. Access means can be provided in the housing to permit replacement of the battery; alternatively the energizing means can be a rechargeable, permanently installed, battery. Use in accordance with the invention of a dispenser requiring connection to an external electrical power source, for example through a transformer and/or converter, is within the presently contemplated scope but will generally be found less convenient than where the energizing means is self-contained within the dispenser.

The actuating means comprises a switch in an electrical circuit connecting the energizing means and the bubble jet device. The switch is suitably finger-operable and can be of any convenient design. Presently preferred is a press-button or trigger activator disposed at a convenient site on the exterior of the housing.

A press-button or trigger activator can have an “off” (non-depressed) position and an “on” (depressed) position; in this case when the activator is depressed the bubble jet device is energized and droplets are dispensed, unless an override is in operation; and when the activator is released it automatically (e.g., by spring-loading) returns to the non-depressed position, electrical power is switched off and droplet dispensing ceases. Thus the amount of liquid dispensed is determined at least in part by the duration of time for which the button or trigger remains depressed. Analogous finger-operable activators include levers, knobs and sliders having an “off” and an “on” position.

Preferably, however, a press-button or analogous activator operates an electrical or mechanical device that switches on the electrical current to the bubble jet device for only a predetermined duration of time to provide a pulse of droplets. Even if the activator remains depressed, no further droplets are generated unless and until the activator is released and pressed again. The duration of the pulse can be controllable, for example by means of a microprocessor as indicated hereinbelow.

The dispenser is preferably of ergonomically appropriate design. Desirably it is small enough to be hand-held but not so small as to present difficulties in use for self-administration by a subject having poor eyesight and/or other disabilities such as arthritic hands. Preferably the dispenser comprises a housing having a droplet delivery aperture; the bubble jet device is located within the housing immediately behind the aperture and oriented such that droplets issuing from the bubble jet device are propelled through the aperture. The standoff (e.g., eye-cup) is typically disposed around the droplet delivery aperture. Access for replacement of a battery or a cartridge reservoir can be afforded by any suitable means, for example by provision of a suitably configured opening in the housing having a removable cover.

The dispenser optionally has a control interface, which can take a form, for example, of one to a plurality of touch pads associated with display means. Any control or data entry elements analogous to touch pads, e.g., a keyboard, or lever or rotary switches, can be used. A preferred display means is a single-line liquid crystal display unit. According to disposition of the control interface, e.g., entry and display of a desired volume and/or rate of delivery of liquid and/or spray pattern (e.g., wide angle, narrow angle or linear jet) the bubble jet device is conditioned, e.g., by a microprocessor circuit, to deliver a selected volume of medicament and/or deliver the medicament at a selected rate. Volume is typically adjusted by adjusting the duration of time for which a pulse of droplet generation by the bubble jet device lasts. Rate is typically adjusted by selecting particular nozzles or combinations of nozzles providing the desired droplet size, or by controlling the repetition rate of droplet emission from a single nozzle. Thus the delivery rate can be selectively high or low depending on the number of nozzles emitting droplets and depending on the repetition rate of droplet emission.

If the bubble jet device is provided with a plurality of nozzles directed at preselected angles to an axis of the dispenser, the spray pattern of droplets can be controlled by selective energizing of thermal resistors.

If desired a microprocessor control circuit can be provided with means to prevent inadvertent excessive use, for example by limiting the maximum dose of liquid which can be applied within a prespecified time period. Also, if desired, the control circuit can be provided with a security lockout that overrides the on/off switch. For example, a programmable security code can be provided such that the bubble jet device is inactivated unless and until a corresponding code is entered by the user.

The dispenser can optionally have electronic interface means, e.g., a plug, jack, socket, connector or transmitter/receiver, that permits the dispenser to interact with an external computer. Such a computer can record data indicative of use, doses dispensed, user identification, patient identification, etc. The computer can also enter data in one or more memories in the control circuit of the dispenser, for example dose to be delivered, spray pattern, security code, etc.

Embodiments of the invention wherein the dispenser is externally controllable by computer are particularly suitable for use in hospitals, clinics, etc., where ophthalmic administration is made to a subject by trained medical personnel. Simpler embodiments are preferred for self-administration by the subject, for example in the subject's home.

According to the method of the invention, a therapeutically effective amount of the medicament is released into the eye from the bubble jet device in not more than a maximum release time as specified herein. The maximum release time is not more than about 1 second, preferably not more than about 0.5 second, more preferably not more than about 0.25 second and most preferably not more than about 0.1 second.

The medicament is a liquid, preferably aqueous, composition comprising at least one drug in dispersed (e.g., dissolved, emulsified or suspended; most preferably dissolved) form. What constitutes a therapeutically effective amount depends on the drug, on the disease or disorder for which the drug is to be administered, on the concentration of the drug in the composition, and on other factors. The term “therapeutically effective amount” herein will be understood to include amounts effective for prevention and/or for treatment of an ophthalmic disease or disorder. Where, as is most frequent, prevention or treatment requires repeated administration of a dosage amount, for example one to several times daily, each dosage amount contributing to effective prevention or treatment is considered herein to be a “therapeutically effective amount”.

Typically the drug is present at a rather dilute concentration in the medicament, for example less than about 5%, more typically less than about 2%, for example less than about 1%. The concentration can be less than about 0.5%, even in some cases less than about 0.2%, in particular cases less than about 0.1%. All concentrations herein, unless otherwise specified, are expressed as weight/volume (w/v). Advantages of a dilute medicament include ease of formulation, storage stability, low viscosity leading to good flow properties, and minimization of any acute injurious effect (e.g., irritation, corrosion, etc.) when placed in the eye.

The low concentration of drug, among other factors, leads to a requirement for delivery of larger volumes of liquid within the maximum release time defined above than were contemplated in above-cited U.S. Pat. No. 5,368,582, which primarily addressed moisturizing and irrigation of the eye. According to the present method, the rate of issuance of droplets from the bubble jet device is not less than about 1 μl s⁻¹, preferably not less than about 5 μl s⁻¹. Excessive rates of issuance should also be avoided, as the eye has limited capacity to retain liquids delivered thereto. Thus the rate of issuance according to the present method is not greater than about 300 μl s⁻¹, preferably not greater than about 50 μl s⁻¹. The total volume of liquid delivered within the maximum release time is not greater than about 50 μl, preferably not greater than about 25 μl, more preferably not greater than about 10 μl.

Illustratively, a dispenser as described herein can be used to deliver 5 μl of the medicament in 1 second at a rate of 5 μl s⁻¹; 7.5 μl of the medicament in 0.5 second at a rate of 15 μl s⁻¹; or 10 μl of the medicament in 0.2 second at a rate of 501 μl s⁻¹.

Any suitable combination of droplet size and number per second can be used to provide the desired rate of issuance. For example, to deliver 5 μl s⁻¹ where droplet size is 1000 picoliters (0.001 μl), 5000 droplets per second must be generated by the bubble jet device. Note that a 1000 picoliter droplet has a diameter of approximately 125 μm.

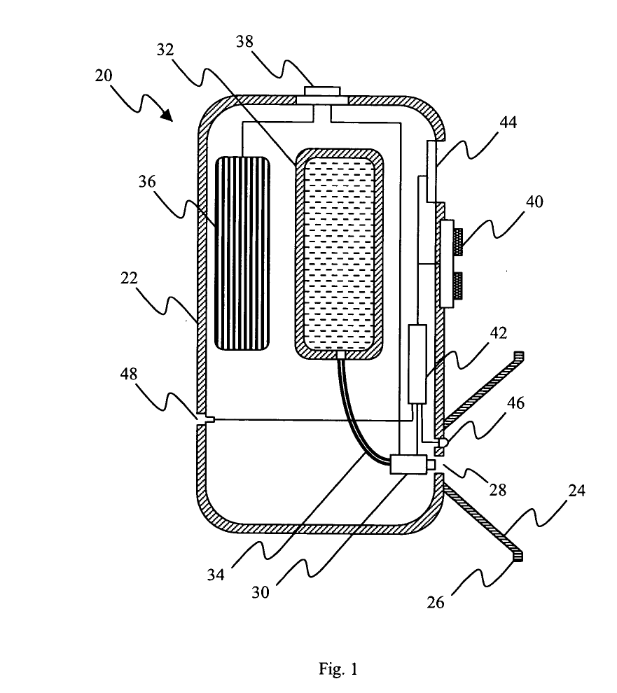

An illustrative dispenser useful in practicing the invention is shown schematically in FIG. 1. The dispenser 20 comprises a hollow housing 22 having attached thereto a standoff, for example an eye-cup 24 with a rim 26 that is configured to engage a circumocular surface. Substantially central to the eye-cup 24 at its locus of attachment to the housing 22 is a droplet delivery aperture 28 in the housing 22. A bubble jet device 30 is disposed within the housing 22 immediately proximal to the droplet delivery aperture 28 and oriented with respect thereto such that droplets issuing from the bubble jet device 30 are propelled through the aperture 28. A refillable or replaceable reservoir 32 is disposed within the housing 22 and is accessible via an opening in the housing having a removable cover (not shown). The reservoir 32 is fluidly connected with the bubble jet device 30 via a conduit 34. The bubble jet device 30 is electrically energized by a battery 36 contained within the housing 22 and accessible via an opening in the housing having a removable cover (not shown). The battery 36 is electrically connected to the bubble jet device 30 via a circuit having an on/off switch, for example a push-button switch 38. A control interface, for example a touch pad unit 40, is optionally provided by means of which a microprocessor 42 is conditioned to control the bubble jet device 30 such that volume, rate and/or spray pattern of the dispensed liquid can be varied. An optional data display unit, for example a liquid crystal display unit 44, displays settings for the bubble jet device and/or other information. An optional sensor 46 located near the droplet delivery aperture 28 detects position and/or orientation of the dispenser with respect to an eye and returns data thereon to the microprocessor 42, for example permitting override control of operation of the bubble jet device 30. Also optionally provided is an electronic interface 48 that enables connection of the microprocessor 42 to an external computer.

Ophthalmic diseases and disorders in which the method of the invention can provide useful treatment and/or prophylaxis include, without limitation, allergic diseases of the eye, for example allergic conjunctivitis, vernal keratoconjunctivitis and eyelid edema; dry eye; keratomalacia; trauma to the eye and adjacent tissues, including conjunctival and corneal foreign body injury, intraocular foreign body injury, contusion and laceration of eyelids, anterior chamber hemorrhage, and thermal and chemical burns of cornea, conjunctiva and eyelids; orbital cellulitis; chronic conjunctivitis; episcleritis; scleritis; superficial punctate keratitis; phlyctenular keratoconjunctivitis; interstitial keratitis; corneal ulcer, including peripheral ulcerative keratitis; uveitis, including iritis, cyclitis, choroiditis, retinitis and any combination thereof, and including uveitis caused by ankylosing spondylitis, Reiter's syndrome, juvenile rheumatoid arthritis, toxoplasmosis, cytomegalovirus, toxocariasis, histoplasmosis, sarcoidosis, tuberculosis and syphilis; Behcet's syndrome; sympathetic ophthalmia; endophthalmitis; exophthalmos; bullous keratopathy; dacryostenosis; acute and chronic dacryocystitis; trichinosis; infective diseases of the eye, for example bacterial (e.g., staphylococcal) blepharitis of ulcerative and seborrheic types, bacterial and viral conjunctivitis (including trachoma and inclusion conjunctivitis), herpes simplex keratitis, and stye; acute retinal necrosis; chalazion; inversion and eversion of eyelids; neoplastic diseases including tumors of eyelids, intraocular tumor and malignant melanoma of choroid; cataract; cyctoid macular edema; birdshot choroidopathy; reticulum cell sarcoma; vascular retinopathies such as arteriosclerotic retinopathy and hypertensive retinopathy; diabetic retinopathy including non-proliferative and proliferative types; macular degeneration including atrophic and exudative types; retinal detachment; retinitis pigmentosa; glaucoma, including primary adult types (e.g., chronic open-angle glaucoma, acute and chronic angle-closure glaucomas, Posner-Schlossman syndrome), congenital (infantile) glaucoma, and secondary glaucoma resulting from pre-existing eye disease such as uveitis, intraocular tumor or cataract; papilledema; papillitis; retrobulbar neuritis; toxic amblyopia; optic atrophy; presbyopia; and ocular motility disorders including cranial nerve palsies.

Classes of ophthalmic drugs that can be delivered by the method of the invention include, without limitation, demulcents; antimycotics, antibacterials, antivirals and other anti-infectives; steroids, NSAIDs, selective cyclooxygenase-2 inhibitors and other anti-inflammatory agents; acetylcholine blocking agents; adrenergic agonists, beta-adrenergic blocking agents, carbonic anhydrase inhibitors, prostaglandins and other antiglaucoma agents; antihypertensives; antihistamines; anticataract agents; and topical and regional anesthetics.

Illustrative specific drugs that can be delivered by the method of the invention are acebutolol, aceclidine, acetylsalicylic acid (aspirin), N⁴ acetylsulfisoxazole, alclofenac, alprenolol, amfenac, amikacin, amiloride, aminocaproic acid, p-aminoclonidine, aminozolamide, anisindione, apafant, atenolol, azithromycin, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cefixime, cefoperazone, cefotaxime, ceftazidime, ceftizoxime, ceftriaxone, celecoxib, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, colistin, cromolyn, cyclopentolate, cyproheptadine, demecarium, dexamethasone, dibucaine, diclofenac, diflusinal, dipivefrin, domeclocycline, dorzolamide, doxycycline, enoxacin, epinephrine, erythromycin, eserine, estradiol, ethacrynic acid, etidocaine, etodolac, etoricoxib, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamicin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levo-bunolol, lidocaine, lonazolac, loteprednol, mafenide, meclofenamate, medrysone, mefenamic acid, mepivacaine, metaproterenol, methacycline, methanamine, methylprednisolone, metiazinic, metoprolol, metronidazole, minocycline, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, parecoxib, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocalne, probenecid, procaine, proparacaine, protizinic acid, pyrimethamine, rimexolone, rofecoxib, salbutamol, scopolamine, silver sulfadiazine, sotalol, sulfacetamide, sulfanilic acid, sulfisoxazole, sulindac, suprofen, tenoxicam, terbutaline, tetracaine, tetracycline, theophyllamine, timolol, tobramycin, tolmetin, travoprost, triamcinolone, trimethoprim, trospectomycin, unoprostone, valdecoxib, vancomycin, vidarabine, vitamin A, warfarin, zomepirac and pharmaceutically acceptable salts, esters and prodrugs thereof.

The method of the invention is illustratively of particular utility in administration to an eye of one or more antiglaucoma agents, such as beta-adrenergic blocking agents, carbonic anhydrase inhibitors and prostaglandins, more particularly PGF_{2α} derivatives. Illustrative beta-adrenergic blocking agents include betaxolol, timolol and salts thereof. Dorzolamide and salts thereof are illustrative carbonic anhydrase inhibitors. Illustrative PGF_{2α} derivatives include latanoprost, travoprost and unoprostone. The method of the invention is useful in administration of such a PGF_{2α} derivative alone or in combination with one or more other drugs. In particular, combinations of a PGF_{2α} derivative such as latanoprost with a beta-adrenergic blocking agent such as timolol can usefully be administered by the method of the invention.

Such antiglaucoma agents are typically ocular hypotensive agents, effective in reducing intraocular pressure whether or not this is manifested as glaucoma. They can also be neuroprotective agents, stopping or retarding progressive damage to nerves resulting from glaucoma or other afflictions. Indications for such drugs, administered by the method of the invention, therefore include, without limitation:
(a) ocular hypertension, including ocular hypertensive episodes following surgery or laser trabulectomy;
(b) congenital glaucoma
(c) open-angle glaucoma
(d) acute angle-closure glaucoma;
(e) chronic angle-closure glaucoma;
(f) secondary glaucoma arising from pre-existing ocular disease, for example inflammatory disease of the anterior segment, uveitis, intraocular tumor, enlarged cataract, central retinal vein occlusion, trauma, operative procedures or intraocular hemorrhage;
(g) retinal vascular diseases, including vasodilation of retinal and choroidal blood vessels;
(h) diabetic retinopathy; and
(i) non-glaucomatous ischemia.

Accordingly, in a preferred embodiment, the composition administered according to the method of the invention comprises an antiglaucoma agent, for example a prostaglandin, illustratively latanoprost, in a dosage amount effective for treatment or prophylaxis of an ophthalmic disease or disorder selected from ocular hypertension, congenital glaucoma, open-angle glaucoma, acute angle-closure glaucoma, chronic angle-closure glaucoma, secondary glaucoma arising from pre-existing ocular disease, retinal vascular diseases, diabetic retinopathy and non-glaucomatous ischemia.

Drugs to be delivered by the present method are first formulated as liquid compositions, that can, if desired, contain more than one drug. Liquid compositions include solutions, suspensions and solution/suspensions. It will be understood that the term “liquid” herein encompasses any composition that is flowable within the dispenser and can be converted to droplets by a bubble jet device as herein contemplated. The drug is dissolved and/or suspended in a carrier liquid that is ophthalmically acceptable to form a composition useful in the method of the invention.

1. A method for treatment or prevention of a disease or disorder of an eye, the method comprising charging a dispenser with a suitable liquid medicament, disposing the dispenser in operative juxtaposition with the eye, and actuating the dispenser to release a therapeutically effective amount of the medicament into the eye; wherein said dispenser comprises (i) an electrically energizable droplet generating device adapted to issue droplets of the liquid medicament at a rate of about 1 to about 300 μl s⁻¹ wherein a therapeutically effective amount of not more than about 50 μl of the medicament is released into the eye in not more than about 1 second, (ii) means for electrically energizing said device, (iii) means for actuating said device, and (iv) a standoff configured to engage a facial surface proximal to the eye and to place the dispenser in operative juxtaposition with the eye.

2. The method of claim 1 wherein said device is a thermal resistor bubble jet device.

3. The method of claim 1 wherein said dispenser further comprises means for selecting prior to actuation thereof at least one of a rate and a duration of issuance of the medicament.

4. The method of claim 3 wherein said selecting means comprises a control interface and display means.

5. The method of claim 4 wherein said control interface comprises a touch pad or means for data entry analogous thereto.

6. The method of claim 4 wherein said display means comprises a liquid crystal display unit.

7. The method of claim 1 wherein the dispenser is charged by installing in a cavity in a housing of the dispenser a replaceable cartridge reservoir containing the medicament; said cartridge reservoir when installed being disposed in fluid connection with a conduit that feeds the medicament from the cartridge to the droplet generating device.

8. The method of claim 1 wherein the standoff of the dispenser is in a form of a cup having a rim contoured to engage a circumocular surface.

9. The method of claim 2 wherein the bubble jet device comprises one to a small plurality of nozzles arranged in close proximity to each other.

10. The method of claim 2 wherein the bubble jet device is (i) disposed within a housing of the dispenser, immediately behind a droplet delivery aperture in said housing; and (ii) oriented whereby droplets issuing from the bubble jet device are propelled through said aperture.

11. The method of claim 10 wherein the standoff of the dispenser is disposed around the droplet delivery aperture.

12. The method of claim 1 wherein a therapeutically effective amount of the medicament is released into the eye from the droplet generating device in not more than about 1 second.

13. The method of claim 1 wherein a therapeutically effective amount of the medicament is released into the eye from the droplet generating device in not more than about 0.5 second.

14. The method of claim 1 wherein a therapeutically effective amount of the medicament is released into the eye from the droplet generating device in not more than about 0.25 second.

15. The method of claim 1 wherein a therapeutically effective amount of the medicament is released into the eye from the droplet generating device in not more than about 0.1 second.

16. The method of claim 1 wherein the medicament is an aqueous composition comprising at least one drug in dispersed form.

17. The method of claim 1 wherein the medicament is an aqueous composition comprising at least one drug in dissolved form.

18. The method of claim 17 wherein the at least one drug is present in the medicament at a concentration of less than about 5% weight/volume.

19. The method of claim 17 wherein the at least one drug is present in the medicament at a concentration of less than about 2% weight/volume.

20. The method of claim 17 wherein the at least one drug is present in the medicament at a concentration of less than about 1% weight/volume.

21. The method of claim 1 wherein the rate of issuance of droplets from the droplet generating device is not less than about 5 μl s⁻¹.

22. The method of claim 1 wherein the rate of issuance of droplets from the droplet generating device is not greater than about 50 μl s⁻¹.

23. The method of claim 1 wherein the total volume of the medicament delivered within a release time of not more than about 1 second is not greater than about 25 μl.

24. The method of claim 1 wherein the total volume of the medicament delivered within a release time of not more than about 1 second is not greater than about 10 μl.

25. The method of claim 1 wherein the disease or disorder is selected from the group consisting of allergic diseases of the eye, dry eye, keratomalacia, trauma to the eye and adjacent tissues, orbital cellulitis, chronic conjunctivitis, episcleritis, scleritis, superficial punctate keratitis, phlyctenular keratoconjunctivitis, interstitial keratitis, corneal ulcer, uveitis, Behcet's syndrome, sympathetic ophthalmia, endophthalmitis, exophthalmos, bullous keratopathy, dacryostenosis, acute and chronic dacryocystitis, trichinosis, infective diseases of the eye, acute retinal necrosis, chalazion, inversion and eversion of eyelids, neoplastic diseases, cataract, cyctoid macular edema, birdshot choroidopathy, reticulum cell sarcoma, vascular retinopathies, diabetic retinopathy, macular degeneration, retinal detachment, retinitis pigmentosa, glaucoma, papilledema, papillitis, retrobulbar neuritis, toxic amblyopia, optic atrophy, presbyopia and ocular motility disorders.

26. The method of claim 1 wherein the disease or disorder is selected from the group consisting of ocular hypertension, congenital glaucoma, open-angle glaucoma, acute angle-closure glaucoma, chronic angle-closure glaucoma, secondary glaucoma arising from pre-existing ocular disease, retinal vascular diseases, diabetic retinopathy and non-glaucomatous ischemia.

27. The method of claim 1 wherein the medicament comprises at least one drug selected from the group consisting of demulcents, antimycotics, antibacterials, antivirals, steroids, NSAIDs, selective cyclooxygenase-2 inhibitors, acetylcholine blocking agents, adrenergic agonists, beta-adrenergic blocking agents, carbonic anhydrase inhibitors, prostaglandins, antihypertensives, antihistamines, anticataract agents, topical anesthetics and regional anesthetics.

28. The method of claim 1 wherein the medicament comprises at least one drug selected from the group consisting of acebutolol, aceclidine, acetylsalicylic acid, N⁴ acetylsulfisoxazole, alclofenac, alprenolol, amfenac, amikacin, amiloride, aminocaproic acid, p-aminoclonidine, aminozolamide, anisindione, apafant, atenolol, azithromycin, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cefixime, cefoperazone, cefotaxime, ceftazidime, ceftizoxime, ceftriaxone, celecoxib, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, colistin, cromolyn, cyclopentolate, cyproheptadine, demecarium, dexamethasone, dibucaine, diclofenac, diflusinal, dipivefrin, domeclocycline, dorzolamide, doxycycline, enoxacin, epinephrine, erythromycin, eserine, estradiol, ethacrynic acid, etidocaine, etodolac, etoricoxib, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamicin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levo-bunolol, lidocaine, lonazolac, loteprednol, mafenide, meclofenamate, medrysone, mefenamic acid, mepivacaine, metaproterenol, methacycline, methanamine, methylprednisolone, metiazinic, metoprolol, metronidazole, minocycline, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, parecoxib, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocalne, probenecid, procaine, proparacaine, protizinic acid, pyrimethamine, rimexolone, rofecoxib, salbutamol, scopolamine, silver sulfadiazine, sotalol, sulfacetamide, sulfanilic acid, sulfisoxazole, sulindac, suprofen, tenoxicam, terbutaline, tetracaine, tetracycline, theophyllamine, timolol, tobramycin, tolmetin, travoprost, triamcinolone, trimethoprim, trospectomycin, unoprostone, valdecoxib, vancomycin, vidarabine, vitamin A, warfarin, zomepirac and pharmaceutically acceptable salts, esters and prodrugs thereof.

29. The method of claim 1 wherein the medicament comprises a PGF_{2α} derivative.

30. The method of claim 29 wherein said PGF_{2α} derivative is selected from the group consisting of latanoprost, travoprost and unoprostone.

31. The method of claim 29 wherein said PGF_{2α} derivative is latanoprost.

32. The method of claim 29 wherein the medicament further comprises a beta-adrenergic blocking agent.

33. The method of claim 32 wherein the beta-adrenergic blocking agent is timolol.

34. An apparatus for use according to the method of any of claims 1 to 33.

35. An apparatus for dispensing a liquid medicament to an eye, the apparatus comprising:
an electrically energizable droplet generating device adapted to issue droplets of the liquid medicament at a rate of about 1 to about 300 μl s⁻¹;
means for electrically energizing said device;
means for actuating said device;
a standoff configured to engage a facial surface proximal to the eye whereby the dispenser can be disposed in operative juxtaposition with the eye; and
means for selecting prior to actuation at least one of a rate and a duration of issuance of the medicament.

36. The apparatus of claim 35 wherein said device is a thermal resistor bubble jet device.
